(19) United States
(12) Patent Application Publication
(10) Pub. No.: US 2014/0322096 A1
(43) Pub. Date: Oct. 30, 2014

(54) SANITIZATION STATION USING PLASMA ACTIVATED FLUID

(71) Applicants: Keith Allen Pelfrey, Wadsworth, OH (US); Robert Gray, Hudson, OH (US); Nick E. Ciavarella, Seven Hills, OH (US); John Peluso, Copley, OH (US); Matt Archer, Aurora, OH (US); Jackson W. Wegelin, Stow, OH (US)

(72) Inventors: Keith Allen Pelfrey, Wadsworth, OH (US); Robert Gray, Hudson, OH (US); Nick E. Ciavarella, Seven Hills, OH (US); John Peluso, Copley, OH (US); Matt Archer, Aurora, OH (US); Jackson W. Wegelin, Stow, OH (US)

(73) Assignee: EP TECHNOLOGIES LLC, Akron, OH (US)

(21) Appl. No.: 14/322,590

(22) Filed: Jul. 2, 2014

Related U.S. Application Data
(62) Division of application No. 13/829,877, filed on Mar. 14, 2013, now abandoned.
(60) Provisional application No. 61/621,078, filed on Apr. 6, 2012.

(51) Int. Cl. A61L 2/18

(52) U.S. Cl. CPC A61L 2/18
USPC 422/291

(57) ABSTRACT

A sanitization station including a fluid source and one or more plasma generators for generating non-thermal plasma is disclosed. One or more nozzles spray a mist or stream of fluid through plasma generated by the one or more plasma generators to activate the fluid. The fluid is then used to sterilize an object. Another sanitization station includes a chamber for holding a fluid and a plasma generator in fluid communication with the chamber for generating plasma. A circulating source moves the fluid in the chamber past plasma generated by the plasma generator to activate the fluid and one or more spray nozzles coat the surface of an object with fluid that is activated by plasma.

### RELATED APPLICATIONS

This non-provisional utility patent application claims priority to and the benefits of U.S. Provisional Patent Application Ser. No. 61/621,078 filed on Apr. 6, 2012 and entitled Sanitization Station Using Plasma Activated Fluid. This application is incorporated herein by reference in its entirety.

### TECHNICAL FIELD

The present invention relates generally to sanitizing systems and more particularly to sanitizing systems using plasma activated fluid for sanitization, such as, for example, pre-surgical hand and arm sterilization and pre-operative sterilization of surgical areas.

### BACKGROUND OF THE INVENTION

Surgeons, nurses and other technicians are required to sterilize their hands and arms prior to operating on patients. Generally, this process requires scrubbing one's hands and arms for a set period of time.

### SUMMARY

A sanitization station including a fluid source and one or more plasma generators for generating non-thermal plasma is disclosed. One or more nozzles spray a mist or stream of fluid through plasma generated by the one or more plasma generators to activate the fluid. The fluid is then used to sterilize an object. Another sanitization station includes a chamber for holding a fluid and a plasma generator in fluid communication with the chamber for generating plasma. A circulating source moves the fluid in the chamber past plasma generated by the plasma generator to activate the fluid and one or more spray nozzles coat the surface of an object with fluid that is activated by plasma. A sanitizing station having a housing having a transparent portion and one or more openings for placing one or more objects within the housing is also disclosed. The sanitizing station also includes a plasma generator for generating a non-thermal plasma and a fluid source connected to an atomizer. The atomizer is positioned to pass atomized fluid through non-thermal plasma generated by the plasma generator to activate the fluid. The activated fluid may be directed to the surface of the one or more objects located within the housing to sanitize the objects.

### BRIEF DESCRIPTION OF THE DRAWINGS

These and other features and advantages of the present invention will become better understood with regard to the following description and accompanying drawings in which:

FIG. 1 is a sanitization station for sanitizing hands, arms and/or objects in accordance with one embodiment of the present invention;

FIG. 2 illustrates another view of the embodiment of FIG. 1;

FIG. 3 illustrates a mister in accordance with one embodiment of the present invention; and

FIG. 4 illustrates another embodiment of a sanitization station.

### DETAILED DESCRIPTION

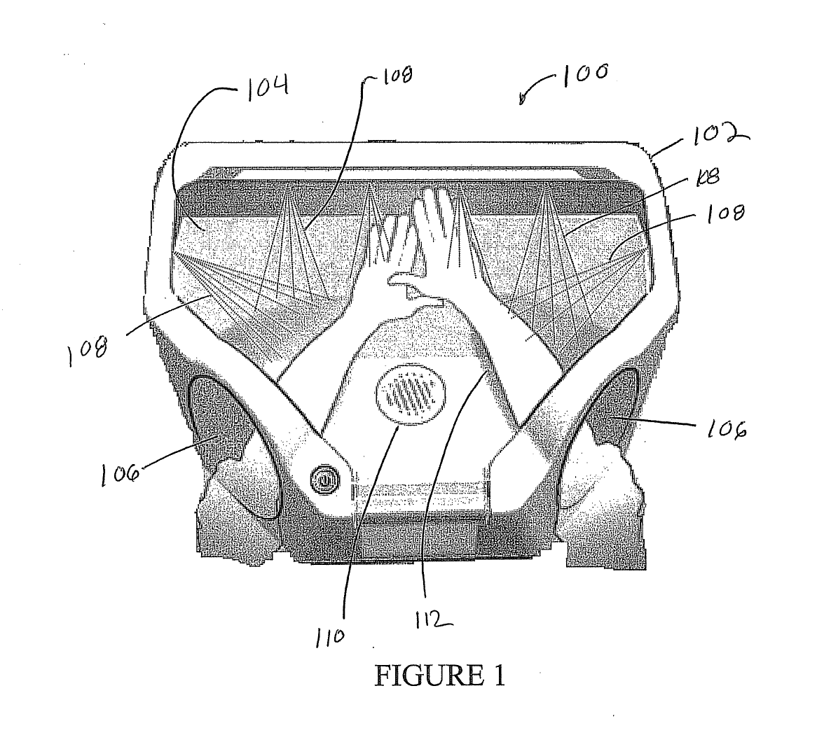

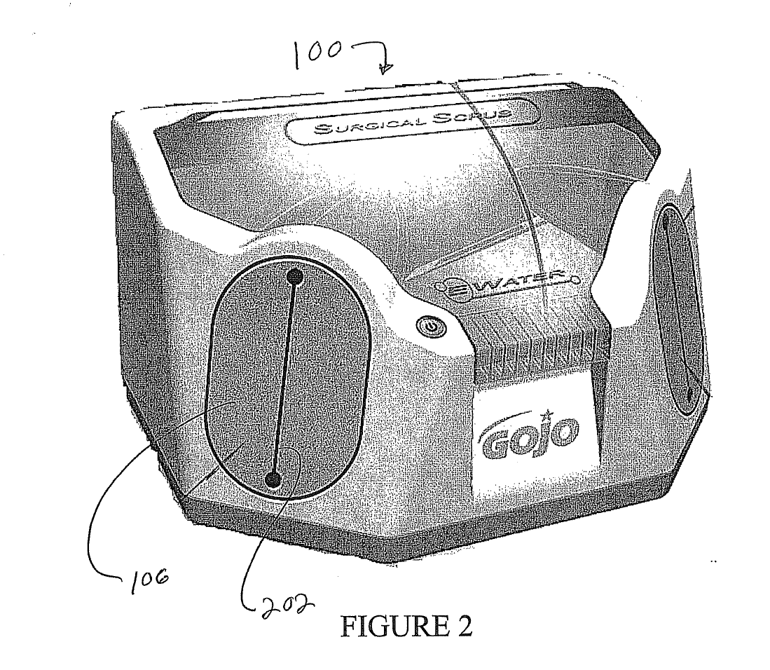

FIGS. 1-2 illustrate a first exemplary embodiment of a sanitizing station 100. Sanitizing station 100 may be used to sanitize objects, such as, for example, hands, arms, objects or equipment. In one embodiment (not shown), sanitizing station 100 may be configured on a larger scale to sanitize a large object, such as an entire body or large piece of equipment. Sanitizing station 100 includes a housing 102. Housing 102 includes a top panel 104. Preferably, top panel 104 is transparent so that a user may see into the sanitizing station. In one embodiment, top panel 104 is made of glass; optionally panel 104 may be made of Plexiglas® or a clear polymer. Housing 102 includes one or more apertures 105. Apertures 105 may have sealing members 106 located therein. Sealing members 106 include slits 202. Slits 202 allow access for a user to place an object, such as hands, arms or equipment within the interior of housing 102. Located on the interior of housing 102 are one or more misters 108. Misters 108 spray a fine mist of plasma activated water onto objects located within housing 102.

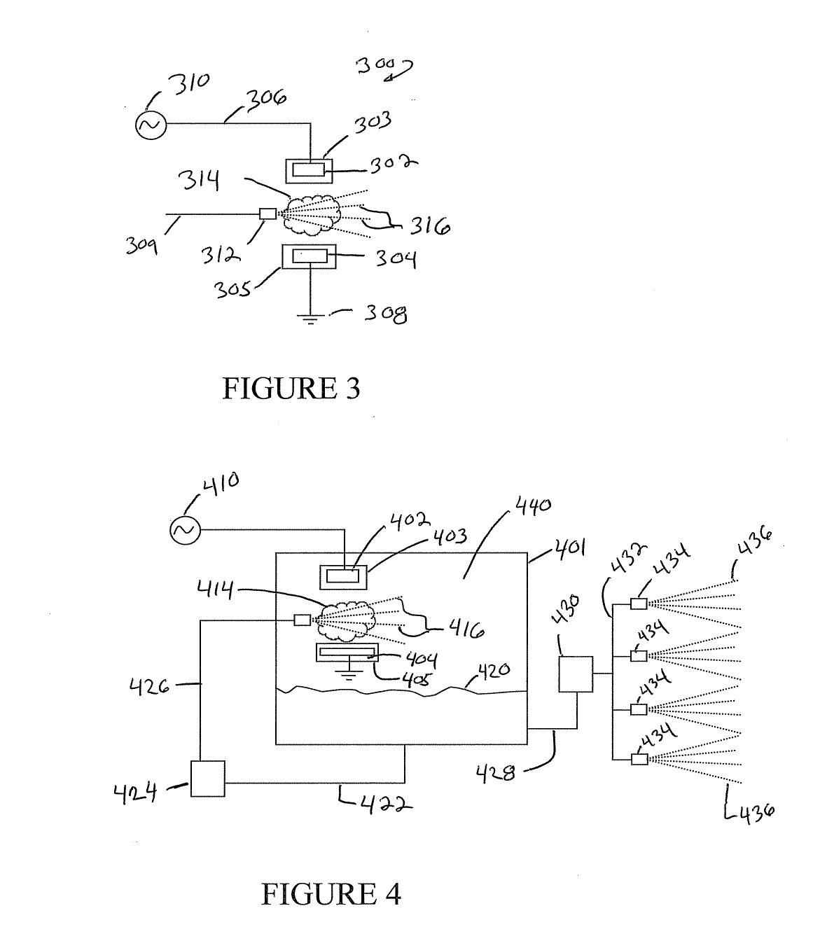

FIG. 3 illustrates an exemplary embodiment of a mister 108 for activating a fluid such as, for example, water, and spraying of plasma activated fluid 316. Mister 108 includes a passage 309 for delivering a fluid such as, for example, water to mister 108. In one embodiment, the fluid is distilled water. An atomizer 312 is located at the end of passage 309. Atomizer 312 atomizes the fluid and directs it between electrodes 302 and 304. In one embodiment, atomizing the fluid creates a large surface area around the small droplets. The large surface area allows for a quicker activation of the fluid.

Electrode 302 is connected to a high voltage source 310 and is surrounded by a dielectric barrier 303. Electrode 304 may also be at least partially surrounded by a dielectric barrier 305. Dielectric barriers 303, 305 prevent arcing between electrode 302 and electrode 304, which is connected to a ground. Dielectric barriers 303, 305 may include, for example, polymers, plastic, glass, ceramics or other known dielectric barriers. High voltage source 310 is connected to electrode 302 by cable 306. High voltage source 310 may have an output of, for example, about 10 kV at between about 0.5 kHz and 500 kHz. In one embodiment, the distance between electrodes 302 and 304 is between about 2 mm and several centimeters.

When electrode 302 is energized, non-thermal plasma is generated between the electrodes 302, 304 by ionizing the gas located between the electrodes 302, 304. Fluid travels under pressure through conduit 309 and through atomizer 312. Atomizer 312 may be, for example, a piezoelectric element, an atomizing nozzle or other mechanism that creates a mist or fine spray of fluid 316. The mist or fine spray of fluid 316 passes through the plasma 314 and becomes plasma activated fluid, such as plasma activated water. The plasma activated fluid 316 is positively charged and is attracted to negatively charged, or grounded, objects such as the hands and arms 112 (FIG. 1). Activated fluid 316 that over sprays objects located within housing 102 collects in the floor of housing 102 and flows out of the drain 110 to be disposed of or cleaned and recycled through sanitizing station 102.

Plasmas, or ionized gases, have one or more electrons that are not bound to an atom or molecule. Plasmas provide high concentrations of energetic and chemically active species and can operate far from thermodynamic equilibrium with high concentrations of active species and yet remain at a temperature that is substantially the same as room temperature.

Non-thermal plasmas, or cold plasmas, contain free electrons. Unlike thermal plasmas, the temperature of the free electrons in non-thermal plasmas is greater than the temperature of the ions and heavy neutral atoms within the plasma. The energy from the free electrons may be transferred to additional plasma components creating additional ionization, excitation and/or disassociation. Fluid that is contacted with plasma becomes “activated” and is referred to herein as plasma activated fluid, and in some embodiments is plasma activated water.

In some embodiments, plasmas may contain superoxide anions [O2.-], which react with H+ in acidic media to form hydroperoxy radicals, HOO., which is a powerful antimicrobial: [O2.-] +[H+]→[HOO.]. Other radical species may include OH. and NO.. Plasma activated water may contain concentrations of one or more of H₂O₂, nitrates, and nitrites.

In addition, the properties of the fluid, such as water, may be altered prior to activation by plasma to increase or decrease concentration of radicals. For example, the pH of water may be adjusted to be acidic or basic. In one embodiment, the pH of the water is between about 2 and 3.5, in another is between about 2 and 3.5, and in yet another is about 2.7. The pH may be adjusted by, for example, adding nitric acid to the water prior to activation. In one embodiment, adjusting the pH levels adjusts the concentrations of radicals allowing for the adjustment of the efficacy of the plasma activated water to kill bacteria.

In addition, the properties of the activated water may be adjusted during the activation process itself by altering the gas that is ionized. For example, the gas that is ionized may be normal air, nitrogen, N₂, Oxygen, O₂, He or combinations thereof

In addition, in one embodiment, additives such as, for example, alcohol may be added to the water prior to activation. The alcohol may be used to increase the efficacy of the activated water for the killing of bacteria. In yet another embodiment, H₂O₂ is used. Preferably, the concentration of H₂O₂ is at about between 1-4%, and more preferably between about 1.5 and 3%.

In one embodiment, co-additives may be added to the water that are capable of stabilizing the antimicrobially active species. Optionally, co-solvents such as low molecular weight alcohols may also be added to the water.

In one embodiment, the water is activated under pressure, resulting in gas being dissolved in the activated water. The activated water may include additives such as, for example, wax that cause an activated foam to form when the liquid is released into normal atmospheric pressure.

FIG. 4 illustrates a sanitizing station 400 in accordance with another embodiment of the present invention. Sanitization station 400 may be used in combination with the components described above with respect to sanitizing station 100 or may be used on its own. Sanitizing station 400 includes a chamber 401. Chamber 401 may be formed by any suitable material such as, for example, polymer. Sanitization station 400 includes a pair of electrodes 402, 404 having dielectric barriers 403, 405. Electrode 402 is connected to a high voltage source 410. Chamber 401 contains a fluid such as, for example, water 420. Other fluids such as H₂O₂, or mixtures of fluid, such as water and alcohol, may be used. Water 420 may be distilled water, tap water, filtered water, water with acidic properties, water with basic properties or water mixed with additives such as, for example, alcohol. Chamber 401 includes a fluid outlet passage 422 connected to a recirculating pump 424. Recirculating pump 424 is connected to fluid passage 426 which is connected to atomizer 412. Atomizer 412 may be a piezoelectric element, an atomizing nozzle or other mechanism that creates a fine mist or spray 416.

The fine mist or spray 416 passes through plasma generated by electrodes 402, 404 when electrode 402 is energized. After passing through the plasma, the fine mist or spray 416 is activated. The activated fine mist or spray 416 is located within chamber 401 and eventually mixes with the water 420 in chamber 401. As the recirculating pump 424 continues to run, the concentration of activated water 420 increases. After a suitable time, enough of water 420 is activated so that the activated water 420 has acquired suitable properties to kill bacteria that it comes in contact with. Recirculating pump 424 may be turned off when the concentration of activated water is suitable to kill the bacteria it contacts, or optionally, recirculating pump 424 may run continuously. Allowing recirculating pump 424 to run continuously may allow for the use of a smaller plasma generator and reduce operating costs.

Chamber 401 includes a second fluid outlet passage 428. Fluid outlet passage 428 is connected to pump 430, which is connected by fluid passage 432 to one or more spray nozzles 434. Spray nozzles 434 may be piezoelectric elements, atomizing nozzles, misting nozzles, etc. When it is desired to sanitize objects such as, for example, hands, arms or equipment, pump 430 is turned on and activated water 420 is sprayed or misted onto the hands, arms and/or other objects. Chamber 401 may also include a water refill passage (not shown) to add additional water to the system.

In one embodiment, chamber 401 is filled with air. Optionally, however, chamber 401 may be filled with other gasses such as, for example, N2, O2 or He; or a combination of one or more of these gases may be used. The gasses may be supplied under atmospheric pressure or under a pressure that is higher or lower than atmospheric pressure. A gas inlet passage (not shown) into chamber 401 may be provided. The use of different gasses may allow tuning of the activated water so that the activated water may have more efficacy killing all bacteria, or may be tuned to have a different efficacy at killing different types of bacteria. For example, it may be beneficial to kill certain bad bacteria while allowing some or all of certain good bacteria to survive.

In one embodiment, a hand held nozzle is used to spray or mist activated fluid on body parts prior to surgery to sterilize the area around where an operation will occur.

In one embodiment, the activated liquid mist has an electrostatic charge that when sprayed on an object having the opposite charge, or a neutral charge, the droplets in the mist rapidly provide uniform coverage over the surface of the object.

In one embodiment (not shown) a closed chamber is used. A plasma gas is created in or directed to the closed chamber, and liquid in the closed container is activated. In one embodiment, an agitator agitates the liquid in the container. In another embodiment, plasma is bubbled up through a liquid to activate the liquid. In still yet another embodiment, an activated liquid is passed by or through a piezoelectric element located at the outlet of a tube to mist or atomize the activated liquid prior to spraying the activated liquid on an object. In one embodiment, a gliding arc plasma generator may be used. The spray of small droplets may pass through the plasma arc, and may require less dwell time to activate the liquid.

In addition, other ingredients may be combined with the fluid to enhance the results of the activated fluid. For example, in one embodiment, a fluid containing a polymer is used. When exposed to the plasma, the polymer cross-links and forms a thin film on the surface of the object, such as skin, being treated. The thin film on the surface of the skin creates longer lasting antimicrobial activity.

While the present invention has been illustrated by the description of embodiments thereof and while the embodiments have been described in considerable detail, it is not the intention of the applicants to restrict or in any way limit the scope of the appended claims to such detail. Additional advantages and modifications will readily appear to those skilled in the art. For example, the sanitizing station 400 may be utilized in a shower, or portable shower system that may be set up to decontaminate persons or large objects on a site that has become contaminated by bacteria. Moreover, elements described with one embodiment may be readily adapted for use with other embodiments. Therefore, the invention, in its broader aspects, is not limited to the specific details, the representative apparatus and illustrative examples shown and described. Accordingly, departures may be made from such details without departing from the spirit or scope of the applicants' general inventive concept.

We claim:

1. A sanitizing station comprising:
a housing;
a fluid source;
one or more plasma generators for generating non-thermal plasma; and
a plurality of nozzles for spraying a mist or stream of fluid through plasma generated by the one or more plasma generators to activate the fluid;
wherein the plurality of nozzles are directed in a plurality of intersecting angles to direct activated fluid to a plurality of surfaces on an object located within the housing.

2. The sanitizing station of claim 1 further comprising a holding tank located in the housing for receiving and holding the activated fluid.

3. The sanitizing station of claim 2 wherein the housing comprises a transparent portion.

4. The sanitizing station of claim 3 wherein the housing comprises one or more openings for moving an object into the housing.

5. The sanitizing station of claim 1 wherein the housing comprises a room.

6. A sanitizing station comprising:
a chamber for holding a fluid;
a plasma generator located in fluid communication with the chamber for generating plasma;
a circulating source for moving fluid in the chamber past plasma generated by the plasma generator for activating the fluid;
one or more spray nozzles for coating the surface of an object with fluid that is activated by plasma.

7. The sanitizing station of claim 6 further comprising a gas source for providing a gas to the chamber.

8. The sanitizing station of claim 7 wherein the gas is selected from air, nitrogen, or oxygen.

9. The sanitizing station of claim 6 wherein the circulating source for moving fluid comprises a recirculating pump and one or more atomizers.

10. The sanitizing station of claim 9 wherein the one or more atomizers comprise one or more piezoelectric elements.

11. The sanitizing station of claim 6 wherein the plasma generator comprises one or more electrodes and a dielectric barrier located between the one or more electrodes.

12. The sanitizing station of claim 1 wherein the fluid is acidic.

13. The sanitizing station of claim 1 wherein the fluid comprises distilled water.

14. The sanitizing station of claim 1 wherein the fluid comprises H₂O₂ at a concentration of between about 1.5 and 3%.

15. The sanitizing station of claim 1 wherein the fluid comprises a polymer that forms a thin film on the object.

16. The sanitizing station of claim 1 wherein the fluid comprises water and an additive to enhance bacterial killing properties of the fluid.

17. A sanitizing station comprising:
a housing having a transparent portion, and one or more openings for placing one or more objects within the housing;
a plasma generator for generating a non-thermal plasma,
a fluid source connected to an atomizer;
the atomizer positioned to pass atomized fluid through non-thermal plasma to activate the fluid;
wherein the activated fluid is directed to the surface of the one or more objects located within the housing.

18. The sanitizing station of claim 17 wherein the atomizer directs the treated fluid to the surface of the one or more objects.

19. The sanitizing station of claim 18 wherein the atomizer comprises a piezoelectric element.

20. The sanitizing station of claim 17 wherein the fluid comprises distilled water.

21. The sanitizing station of claim 17 wherein the plasma generator ionizes nitrogen.

22. The sanitizing station of claim 17 wherein the plasma generator ionizes air.

23. The sanitizing station of claim 17 further comprising a chamber for activating the fluid and wherein the chamber is configured to hold a quantity of activated fluid.
